(19) United States
(12) Patent Application Publication
(10) Pub. No.: US 2011/0313313 A1
(43) Pub. Date: Dec. 22, 2011

(54) Device for Detecting Skin Sensitivity and Use of the Same

(75) Inventor: Andrew Levy, Bristol (GB)

(73) Assignee: University Hospitals Bristol NHS Foundation Trust, Bristol (GB)

(21) Appl. No.: 13/059,214

(22) PCT Filed: Aug. 14, 2009
(86) PCT No.: PCT/GB09/01993

§ 371 Date: May 9, 2011

(30) Foreign Application Priority Data
Aug. 15, 2008 (GB) 0814968.4

(51) Int. Cl. A61B 5/00

(52) U.S. Cl. 600/552

(57) ABSTRACT

A device for determining the sensitivity of the skin of a subject is provided, the device comprising a body having a head for placing in contact with the skin of the subject; vibration means for imparting a vibration to the head; a switch assembly for activating the vibration means; wherein the switch assembly is operable by a user applying pressure to the switch assembly in a direction that does not apply pressure to the skin of the subject. A method of using the device is also disclosed.

The present invention relates to a device for detecting skin sensitivity, in particular a device for applying vibrations to the skin of a subject, and to the use of the same.

Several conditions are known to afflict humans that result in a reduction or loss of sensitivity of the skin in parts of the body. For example, subjects with diabetes often experience a decrease in sensitivity of the skin of extremities of the body, in particular fingers and hands, and toes and feet. Other causes of a loss of sensitivity in the skin of a subject include conditions adversely affecting the function of the brain and/or nerves of the subject. A proper diagnosis and treatment of a subject requires that the level of loss of sensitivity in the relevant parts of the body is assessed and quantified.

Devices for making such an assessment are known in the art and are currently in use by clinicians and medical staff.

U.S. Pat. No. 2,704,539 is concerned with a skin sensitivity detector. The detector comprises a generally cylindrical body housing a plunger and a spring. A needle extends from one end of the body and may be depressed inwards within the body against the action of the spring. The detector is arranged to allow the spring to be set with a predetermined resistance to movement of the needle. In use, the needle is pressed against the skin of the subject and the pressure applied determined according to the preset resistance of the spring. The device of U.S. Pat. No. 2,704,539 is suitable for inducing a quantifiable level of pain in the subject, to determine certain aspects of the subjects level of sensitivity.

The needle in the detector of U.S. Pat. No. 2,704,539 is fixed, with the exception of being depressible within the body against the spring. An alternative method for assessing the sensitivity of a subject's skin is to use a vibrating member, which can be applied to the skin to determine the level of sensation felt by the subject. The sensitivity of a subject to vibration is a different sensation to that of pain, such as induced by the device of U.S. Pat. No. 2,704,539, and a measure of the ability of the subject to detect vibrations is a valuable tool in the diagnosis of a range of conditions. U.S. Pat. No. 5,433,211 discloses a method and system for assessing the vibrotactile perception of a subject. The system comprises a so-called ‘mini-shaker’ which is applied to the skin of the subject at the site of interest. The system applies a number of frequencies of vibration to the skin by means of the mini-shaker and the subject indicates their response using a switch. The data relating to the applied frequencies and the response signals from the subject are processed to provide an indication of the threshold levels of the sensitivity of the subject's skin. It will be appreciated that the system of U.S. Pat. No. 5,433,211 is complex to construct and operate.

A vibrator for diagnosing joint disorders is disclosed in U.S. Pat. No. 5,458,119. The device is hand-held and comprises a handle housing a vibration means. A detachable applicator is connected to the handle and, in use, is applied to the region of the subject under investigation. The device produces pain in the subject by applying vibrations to the spinous process above a degenerated disc, thereby allowing the condition of the subject to be assessed. The tip of the applicator that is applied to the skin of the subject may take a range of forms, including smooth and flat, smooth and arcuate, a tapered form, and a needle for invasive investigations. The arcuate tip is shown as being domed and substantially hemispherical. The device is activated by means of a switch located in the handle, which is moved longitudinally between the ‘on’ and ‘off’ positions. The device of U.S. Pat. No. 5,458,119 generally applies a pattern of vibrations to a relatively large area of the skin of the subject, with the intention of targeting the bones and joints beneath the skin with vibrations. This renders the device unsatisfactory for an accurate assessment of the sensitivity of the skin of the subject in specific target areas, which may be required to assess localised nerve damage to extremities such as fingers and toes.

More recently, a device for the detection of neuropathy is disclosed in U.S. Pat. No. 5,931,793. The device, intended to be pocket sized, comprises a generally cylindrical housing, from one end of which extends a stimulus head or probe which is caused to vibrate. The vibrations are provided by a vibration device including an electric motor rotating and eccentric weight and powered by batteries located within the housing. In one embodiment, the vibration device is housed within the stimulus head. In an alternative embodiment, the vibration device is disposed at one end of the housing, with the stimulus head extending from the other end of the housing. The device is activated by means of a switch assembly having a sliding switch member that is moved longitudinally along the housing by the user between an ‘on’ position and an ‘off’ position. The stimulus head shown in U.S. Pat. No. 5,931,793 has two forms. In the first embodiment, the stimulus head is a cylindrical member having a flat end. In the second embodiment, the stimulus head is a cylindrical member having a domed end. It has been found that the forms of the stimulus head shown in U.S. Pat. No. 5,931,793 limit the available angle at which the device may be applied to the skin of the subject in order to provide an accurate assessment and avoid false responses from the subject.

A marking device with a vibrating yieldable tip is shown and described in U.S. Pat. No. 5,208,987. The device is operated by a longitudinally sliding switch on the side of the device. There is no suggestion that this device is suitable for determining the skin sensitivity of a subject.

U.S. Pat. No. 4,881,526 discloses an intra-vaginal probe for applying electrical and mechanical stimulations to a female subject for controlling urinary incontinence.

There is a need for an improved vibration device for use in assessing the sensitivity of the skin of a subject in small, localised regions of the skin, in particular for use in providing and accurate and repeatable assessment of the sensitivity of the skin of the extremities of the subject, such as the fingers and toes. In addition, the device must be convenient to use, while at the same time allowing the user to mask or cover the device being used, so as to avoid the subject gaining an indication of the apparatus being used by visual or other means.

In a first aspect, the present invention provides a device for determining the sensitivity of the skin of a subject, the device comprising:

a body having a head for placing in contact with the skin of the subject;

vibration means for imparting a vibration to the head;

a switch assembly for activating the vibration means;

wherein the switch assembly is operable by a user applying pressure to the switch assembly in a direction that does not apply pressure to the skin of the subject.

An important aspect of assessing the skin sensitivity of a subject is for a user to be able to apply the required level of mechanical stimulation, in particular vibration, to the skin. In many cases the level of vibration required to be applied is particularly low, in order to be able to assess a minor loss of sensitivity, for example when attempting to identify the early stages or the onset of a condition affecting the nerves of the subject. In such cases, it is important to avoid alerting the subject to the presence of the device, in order to avoid the subject giving a false positive response. Such false positive responses can mask the presence of damage to nerves and small reductions in the sensitivity of the skin that occur in the early stages of particular conditions. This in turn can prevent the early diagnosis of the condition of the subject.

It has been found that the mere act of the user switching on a device can be felt by a subject, even though the subject cannot feel the subsequent vibrations, giving rise to a false response during the assessment. This is particularly the case with the devices of the prior art that rely upon a switch assembly having a longitudinally moving member. As the switch is operated, pressure is applied through the device to the user. The device of the present invention overcomes this problem by providing a switch assembly that is operable by the user without having the operation of the switch applying pressure to the skin of the subject. In this way, the device of the present invention may be applied to the skin of a subject and then turned on and off as required, without providing mechanical stimulation to the skin of the subject other than that generated by the vibration means. This in turn significantly reduces the number of false positive responses from the subject and gives rise to a significant improvement in the accuracy of the assessment.

The device of the present invention comprises a body. The body most preferably houses all the components of the device, including the vibration means, its power source, and the switch assembly. The body may have any suitable shape and size. Most preferably, the body is of a size and shape that it can be hand-held and can be carried in the pocket of a clinician or other medical practitioner. The shape of the body should allow it to be held securely, yet gently by the user, in order to avoid apply other mechanical stimuli to the skin of the subject when in use. In particular, the body is of a shape and size that it can be held and cradled in one hand of the user without being seen by the subject and the device operated discreetly using just the one hand. In this way, a particular vibrational stimulus may be applied to the subject without the subject being aware of an obvious means of generating the stimulus. This allows a very specific answer and reply to the test to be obtained from the subject, in turn allowing a more accurate assessment of the subject to be made.

The body of the device comprises a head. In use, the head is placed against the skin of the subject in the region under investigation. The mechanical stimuli generated by the device are transmitted to the skin of the subject through the head. The head may have any suitable shape and configuration that allows the mechanical vibrations to be transmitted to the skin of the subject. It is preferred that angular, pointed or sharp forms are avoided for the head, as these may result in skin of the subject receiving mechanical stimuli other than just the vibrations and make the device more difficult to use. Most preferably, the head has a rounded or arcuate surface for contacting the skin. The surface of the head is most preferably smooth. However, the surface of the head may be provided with facets or countours.

In a particularly preferred embodiment, the head has a rounded, arcuate surface. As noted above, such rounded and arcuate forms are known in the art. However, the known devices, if they employ a rounded member, generally have a form that is arcuate no more than hemispherical. As noted above, this has been found to restrict the range of angles at which the device can be applied to the skin of the subject without impairing or reducing its effectiveness. It has been found that a head that has an arcuate or curved surface, a plurality of intersecting arcs of which extend through an angle greater than 180° provides for a significant improvement in the ease and range of use of the device.

Accordingly, in a further aspect, the present invention provides a device for determining the sensitivity of the skin of a subject, the device comprising:

a body having a head for placing in contact with the skin of the subject; and

vibration means for imparting a vibration to the head;

wherein the head comprises an arcuate surface, a plurality of arcs of which extend through an angle greater than 180°.

The head has an arcuate surface having a plurality of arcs extending through an angle greater than 180°. The head preferably has an arcuate surface with arcs extending through an angle of greater than 230°, more preferably at least 270°. The arcuate surface may take any form, but is preferably regular. It is particularly preferred that the surface of the head is a portion of a sphere, which portion is greater than a hemisphere. In one preferred embodiment, the head is substantially spherical, allowing a particularly high range of angles of application of the device to the skin of a subject. The head is preferably mounted on a generally elongate portion of the body, such that it can extend from the hand of the user when being held.

The head may be arranged to be detachable from the body. In a particularly preferred embodiment, the head is formed, integrally with the body.

The head of the device is preferably provided with a surface that is easily cleaned for reasons of hygiene. Alternatively, or in addition thereto, the head may be provided with a cover or coating, preferably removable or replaceable, again for the purposes of hygiene. The surface of the cover is preferably smooth, but may be faceted or contoured, as desired.

The device comprises a means for causing the head to vibrate, which vibrations are communicated to the skin of the subject during use. The head may be arranged to vibrate independently of the body and the rest of the device. However, for simplicity and ease of construction and use, it is preferred that the vibration means, when operated, cause the entire device to vibrate. This can also provide the user with a direct indication of the nature of the vibrational stimuli being applied to the subject. Any suitable means for generating vibrations in the head of the device can be employed. One particularly suitable means comprises an electric motor which, when activated, rotates an eccentrically mounted weight.

The vibration means may be arranged to generate vibrations of a single frequency and amplitude. For example, in the aforementioned embodiment, the electric motor may be arranged to rotate at a single speed when activated. Alternatively, the vibration means may be arranged to provide vibrations at different frequencies, as required by the user, for example by using a variable speed motor.

The vibration means are most preferably housed within the body, together with their power source, for example batteries, as required. The power source is preferably a single battery or cell. Suitable batteries with sufficient power to drive the vibration means yet small enough to be housed within a small, discrete body, as described above, are known in the art and commercially available.

In a preferred embodiment, the device is provided with means to attenuate the vibrations passing from the head to the skin of the subject. This is particularly the case where the vibration means are arranged to provide vibrations at a single frequency and amplitude. By the provision of attenuating means, the vibrations of the device may be reduced as required, for example to obtain a more accurate assessment of the level of sensitivity of the subject's skin.

Accordingly, in a further aspect, the present invention provides a device for determining the sensitivity of the skin of a subject, the device comprising:

a body having a head for placing in contact with the skin of the subject;

vibration means for imparting a vibration to the head; and

means for modifying the frequency and/or amplitude of the vibrations of the head be passed to the skin of the subject.

The means for modifying the vibrations may be any suitable means that amplify or attenuate the vibrations. In one preferred embodiment, the means are attenuating means. The means for modifying the vibrations may be a layer of material, for example a layer of attenuating material, applied to the surface of the head of the device. The layer of material may extend across all or a portion of the surface of the head. Suitable materials for modifying the vibrations of the head to pass to the skin of the subject include rubbers, elastomers and other polymers. A particularly suitable material is silicone rubber.

The attenuating means may be provided as a permanent layer of material applied to the surface of the head. Alternatively, and more preferably, the layer of material is in the form of a removable cover that may be applied to the head, when attenuation of the vibrations is required. The cover may act as a hygienic barrier, as described hereinbefore. The surface of the layer of material or cover is preferably smooth, but may be faceted or contoured.

In one embodiment, the head of the device is provided with a layer of material for modifying the vibrations of the head in the form of a cover, the cover having a probe portion extending therefrom. In use, the distal end of the probe portion may be placed in contact with the skin of the subject. The probe portion may have any suitable shape, for example cylindrical.

The device comprises a switch assembly for activating the vibration means. Any suitable switch assembly may be employed. In the device of the first aspect of the present invention, the switch assembly is arranged to be activated by the user in a way that does not require the user to apply pressure to the head of the device and to the skin of the subject. In this way, the device may be activated while the head is in contact with the skin of the subject, allowing for the repeated application of vibrational stimuli to the skin of the subject without requiring the user to repeatedly remove and replace the head of the device in contact with the skin. The switch assembly is most preferably one that can be held by the user in one hand and operated by user with the same hand.

The switch assembly will require the user to apply a force to the device to switch the device on and/or off. The switch assembly is of an arrangement that any force applied by the user to the device is applied in a direction other than directly towards the skin of the subject when the device is held with the head in contact with the subject's skin. In particular, the switch assembly is preferably arranged such that the force applied by the user to operate the switch assembly is applied laterally, that is very obliquely or substantially parallel to the surface of the skin of the subject. In a preferred embodiment, the switch assembly comprises a switch member that is pressed by the user. The switch member is most preferably arranged such that it can be operated by the user squeezing the body of the device. The switch assembly may be arranged such that the user must apply pressure both to switch the device on and switch it off. In a preferred arrangement, the switch assembly is arranged to have the switch member biased in the ‘off’ position, with the user applying force to move the switch member into the ‘on’ position and activate the device. Any suitable biasing means may be used. In a particularly preferred embodiment, the switch member comprises a flexible, resilient plate that may be depressed by the user to activate the vibration means, the plate returning to its rest position when released by the user and deactivating the vibration means.

In a further aspect, the present invention provides a method for assessing the sensitivity of the skin of a subject comprising applying to the skin of the subject a device as hereinbefore described.

In particular, the present invention provides a method for assessing the sensitivity of the skin of a subject comprising: applying to the skin a device comprising a body and a head, the device being applied such that the head contacts the skin of the subject;

activating the device by applying a force to the device that extends laterally of the skin of the subject, such that activation does not require a force to be applied to the head of the device in the direction towards the skin of the subject, the head applying a vibrational stimulus to the skin of the subject when the device is activated; and

monitoring the response of the subject to the vibrational stimulus applied.

An embodiment of the present invention will now be described, by way of example only, having reference to the accompanying drawings, in which:

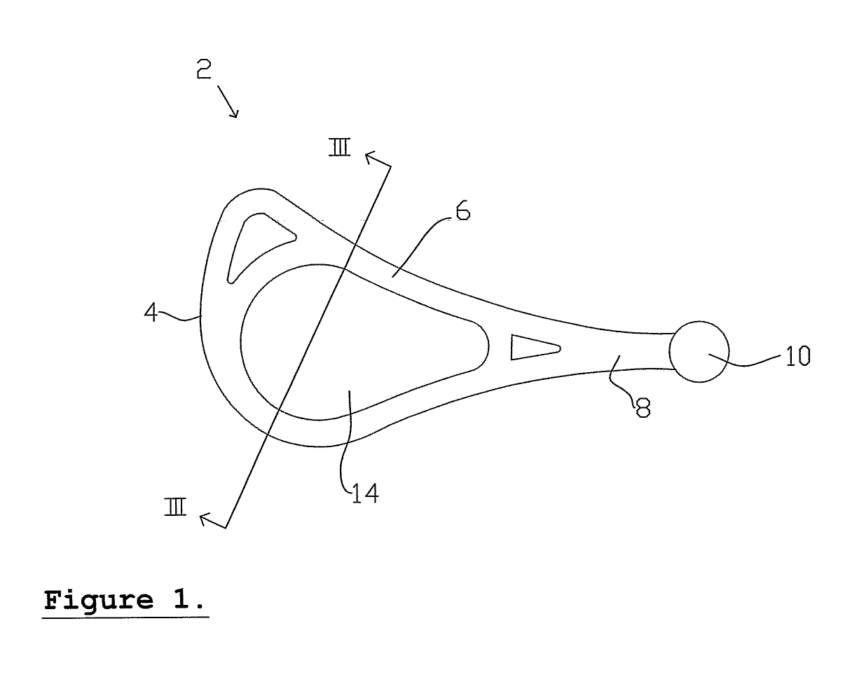

FIG. 1 is a side view of a device according to one embodiment of the present invention;

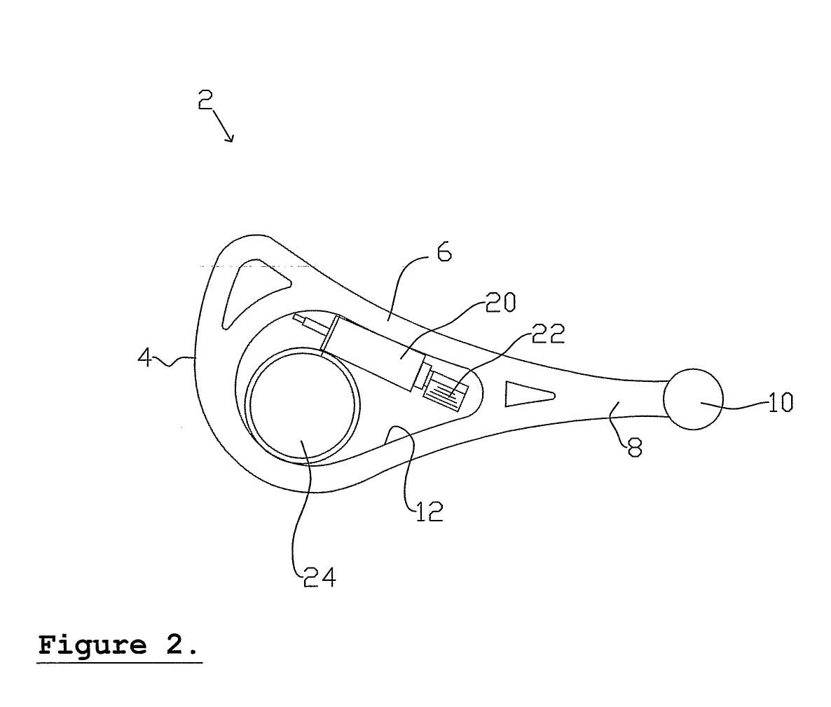

FIG. 2 is a side view of the device as shown in FIG. 1 with the cover removed to show the internal components of the device;

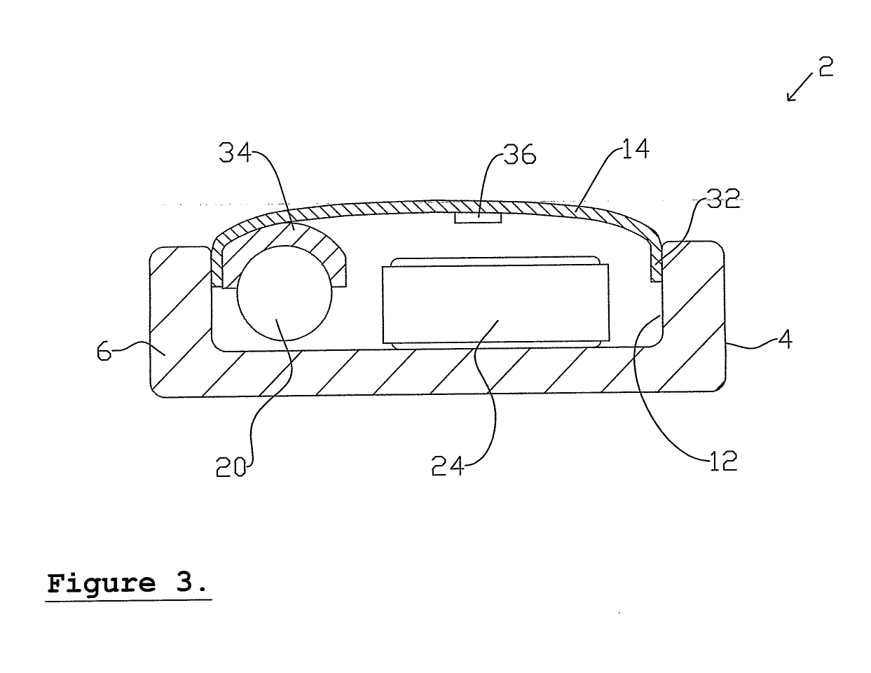

FIG. 3 is a cross-sectional view of the device along the line III-III of FIG. 1;

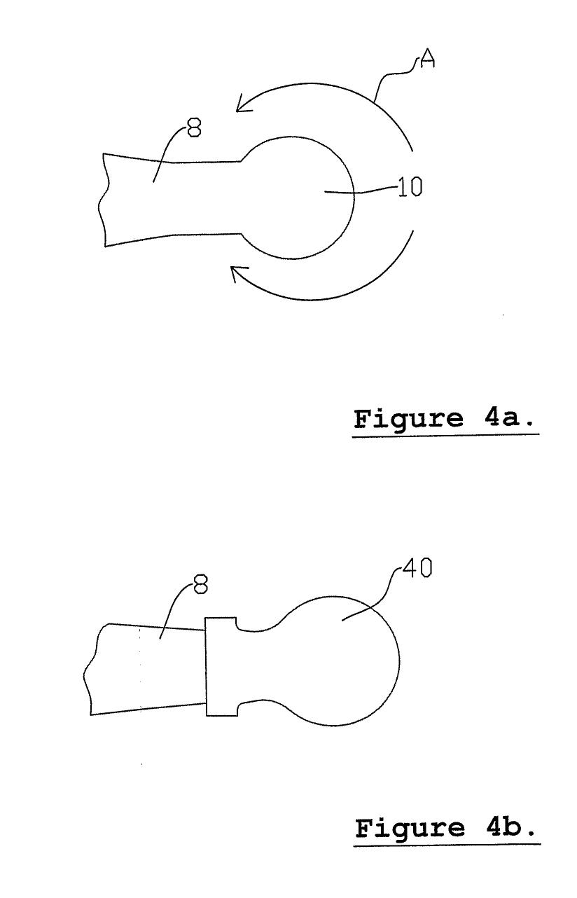

FIG. 4a is an enlarged view of the head portion of the device of FIG. 1; and

FIG. 4b is a view of the head portion of FIG. 4a with an attenuating cover in place.

Referring to FIG. 1, there is shown a device, generally indicated as 2, for assessing the skin sensitivity of a subject. The device 2 has a body 4 having a main body portion 6 and an elongate body portion 8 extending from the main body portion 4 and terminating in a generally spherical head 10. The main body portion 6 is of a shape and configuration that allows it be held by the user in one hand, with the elongate body portion 8 extending from the hand leaving the head 10 unobstructed to be applied to the skin of the subject.

The head 10 is generally spherical in shape and is shown in larger detail in FIG. 4a. In particular, the head can be considered to have a curved surface having an arc when viewed from the side, as in FIG. 1, represented by the line A in FIG. 4a, extending through an angle of greater than 270°. In this respect, it is most advantageous that the head has a form that has a curved surface that is a portion of a sphere greater than a hemisphere.

The head 10, by being generally spherical, allows the device to be applied to the skin of the subject throughout a wide range of angles. The head 10 has a diameter larger than the width of the elongate body portion 8, further increasing the range of angles of application of the device.

The main body portion 6 is provided with a cavity 12 for housing the components of the device, as shown more clearly in FIG. 2. A cover 14 extends over the cavity 12 and serves as a switch member, as described in more detail hereinbelow.

Disposed within the cavity 12 of the device 2 is an electric motor 20 of generally conventional configuration. The motor 20 has a shaft extending therefrom. A weight 22 is eccentrically mounted in the shaft, such that rotation of the shaft and weight generates a vibration. The cavity 12 further houses a generally disc-shaped battery 24. The battery 24 and motor 20 are connected by a suitable cable (not shown for clarity).

Referring to FIG. 3, there is shown a cross-section through the device of FIG. 1, showing the arrangement of the cover 14. As shown, the cover 14 comprises a steel plate 30 having a flange 32 extending from its edge. The flange 32 is an interference fit within the cavity 12 of the body 4 and provides the means for holding the cover 14 in place on the body. Alternative means for holding the cover 14 in place on the body may be employed. For example, the cover 14 may be secured over the cavity by a suitable adhesive or sealant. A further alternative is to provide the cover with one or more flexible extensions for engaging with indents in the inner surface of the cavity.

The steel plate 30 is generally flat having a domed central portion. The cover 14 comprises a clip 34 for retaining the motor 20, with the body of the electric motor being in electrical contact with the clip and the cover 14. A contact 36 is disposed centrally on the inner surface of the plate 30 to lie over the battery 24. In the rest position, shown in FIG. 3, the contact 36 is held out of contact with the battery by the domed form of the steel plate 30. Depression of the domed plate 30 against its natural bias causes the contact 36 to contact the battery 24, completing the circuit with the motor 20 and providing power from the battery to the motor. Releasing the pressure against the plate 30 allows it to return under its natural bias to the rest position with the contact 36 removed from the battery. As an alternative design, the contact 36 may be omitted and the domed plate 30 free to be depressed sufficiently for the surface of the plate to contact the battery 24.

In use, the device 2 is held in one hand of the user and applied to the skin in the target area of the subject, such that the head 10 is in contact with the surface of the skin. As noted hereinbefore, due to the curved form of the head, the angle at which the device is applied to the skin is not critical. The user presses the outer surface of the domed steel plate 30 of the cover 14, pushing the contact 36 into contact with the battery, thereby activating the motor 20. The motor rotates the eccentric weight 22, causing the entire device to vibrate. The frequency of the vibration is determined by the speed of rotation of the motor. The vibration is transmitted by the head 10 to the skin of the subject. The subject is asked to indicate whether the vibrations can be detected in the target area. When the user releases the pressure on the steel plate 30, the contact moves away from the battery, opening the circuit and stopping the motor. The user may repeatedly switch the device on and off by pressing and releasing the plate without needing to remove the device from the skin of the subject. As the force applied to the plate is sideways or lateral, there is no force applied through the head of the device to the skin of the subject when the device is activated. This reduces the tendency of the subject to detect the device and give a false indication during the assessment.

Referring to FIG. 4b, there is shown the head 10 of the device with an attenuating cover 40 applied to the outer surface of the head. The attenuating cover 40 is formed from silicone rubber and extends over the entire surface of the head 10. The attenuating cover 40 acts to absorb vibrations of the device and limit their transmission from the head to the skin of the subject. In this way, the amplitude of the vibrations reaching the skin of the subject modified. The attenuating cover may be applied to increase the sensitivity of the examination of the subject. The cover may be removed from the head and replaced with a similar cover for use with the next subject, thus maintaining the hygiene of the device. The device may be provided with a series of covers having attenuating material of different densities and thicknesses, to allow the degree of attenuation to be varied. Alternative materials may be employed to form the cover, thereby altering the change in the vibrations.

While the device as hereinbefore described in both general and specific terms is of particular use in the assessment of the sensitivity of the skin of a subject, it will be appreciated that the device may be used in a wider range of applications where vibrational stimuli to parts of the body of the subject are required.

### EXAMPLE

An experiment was conducted into the response of 100 subjects to the application of a device as shown in the accompanying figures and described in detail above. The device was applied to the skin of a foot of each subject and the subject asked to indicate whether vibrations could be detected. The device was activated once the head of the device was in contact with the skin of the subject.

The experiment was repeated on the same 100 subjects using a standard tuning fork, as used by medical practitioners to determine the presence of neuropathy in subjects. Again, the stem of the tuning fork was applied to the skin of a foot of each subject. The tuning fork was struck before being applied.

Of the 100 subjects, 55 subjects detected the presence and vibration of the device of the present invention, while 45 subjects could not detect the device when activated. These results were consistent with diagnoses of the subjects as being sufferers of a significant degree of neuropathy.

When using the tuning fork, as similar response was obtained from the subjects. However, a number of subjects indicated that the tuning fork could be detected if struck heavily, indicating some propagation of vibrations from the sight of application of the tuning fork along the bones of the subject to areas with less or no neuropathy. No similar effect was detected from the device of the present invention.

In summary, the device of the present invention provided an indication of neuropathy consistent with the indications provided by the standard techniques in the art using a tuning fork. However, the device of the present invention did not give the false indications occurring on a number of occasions with the tuning fork.

1. A device for determining the sensitivity of the skin of a subject, the device comprising:
a body having a head for placing in contact with the skin of the subject; and
vibration means for imparting a vibration to the head;
wherein the head comprises an arcuate surface, a plurality of arcs of which extend through an angle greater than 180°.

2. The device according to claim 2, wherein the device is hand held.

3. The device according to claim 3, wherein the device may be held in and operated by a single hand of the user.

4. The device according to any of claims 1 to 3, wherein the head has an arcuate surface with arcs extending through an angle greater than 230°.

5. The device according to claim 4, wherein the head has an arcuate surface with arcs extending through an angle greater than 270°.

6. The device according to any of claims 1 to 5, wherein the surface of the head is a portion of a sphere greater than a hemisphere.

7. The device according to claim 6, wherein the head is substantially spherical.

8. The device according to any of claims 1 to 7, wherein the body comprises an elongate portion, the head being located at the distal end of the elongate portion.

9. The device according to any of claims 1 to 8, wherein the head is integral with the body.

10. . The device according to any of claims 1 to 9, wherein the head is provided with a cover or coating.

11. The device according to claim 10, wherein the cover or coating is removable.

12. The device according to either of claim 10 or 11, wherein the cover or coating is formed from a material that modifies the vibrations transmitted by the head to the skin of the subject.

13. The device according to any of claims 1 to 12, wherein the vibration means, when activated, causes the entire device to vibrate.

14. The device according to any of claims 1 to 13, wherein the vibration means comprises an electric motor which, when activated, rotates an eccentrically mounted weight.

15. The device according to any of claims 1 to 14, further comprising a switch assembly, wherein the switch assembly is arranged to be activated by a lateral force applied by the user.

16. The device according to claim 15, wherein the switch assembly comprises a switch member to be pressed by the user.

17. The device according to claim 16, wherein the switch member is biased to the off position.

18. The device according to claim 17, wherein the switch member is a flexible, resilient plate.

19. A device for determining the sensitivity of the skin of a subject, the device comprising:
a body having a head for placing in contact with the skin of the subject;
vibration means for imparting a vibration to the head;
a switch assembly for activating the vibration means;
wherein the switch assembly is operable by a user applying pressure to the switch assembly in a direction that does not apply pressure to the skin of the subject.

20. The device according to claim 19, wherein the body houses the vibration means and the switch assembly.

21. The device according to either of claim 19 or 20, wherein the device is hand held.

22. The device according to claim 21, wherein the device may be held in and operated by a single hand of the user.

23. The device according to any of claims 19 to 22, wherein the head has a rounded or arcuate surface for contacting the skin of the subject.

24. The device according to claim 23, wherein the head has an arcuate surface having a plurality of arcs extending through an angle greater than 180°.

25. The device according to claim 24, wherein the head has an arcuate surface with arcs extending through an angle greater than 230°.

26. The device according to claim 25, wherein the head has an arcuate surface with arcs extending through an angle greater than 270°.

27. The device according to any of claims 24 to 26, wherein the surface of the head is a portion of a sphere greater than a hemisphere.

28. The device according to claim 27, wherein the head is substantially spherical.

29. The device according to any of claims 19 to 28, wherein the body comprises an elongate portion, the head being located at the distal end of the elongate portion.

30. The device according to any of claims 19 to 29, wherein the head is integral with the body.

31. The device according to any of claims 19 to 30, wherein the head is provided with a cover or coating.

32. The device according to claim 31, wherein the cover or coating is removable.

33. The device according to either of claim 31 or 32, wherein the cover or coating is formed from a material that modifies the vibrations transmitted by the head to the skin of the subject.

34. The device according to any of claims 19 to 33, wherein the vibration means, when activated, causes the entire device to vibrate.

35. The device according to any of claims 19 to 34, wherein the vibration means comprises an electric motor which, when activated, rotates an eccentrically mounted weight.

36. The device according to any of claims 19 to 35, wherein the switch assembly is arranged to be activated by a lateral force applied by the user.

37. The device according to claim 36, wherein the switch assembly comprises a switch member to be pressed by the user.

38. The device according to claim 37, wherein the switch member is biased to the off position.

39. The device according to claim 38, wherein the switch member is a flexible, resilient plate.

40. A device for determining the sensitivity of the skin of a subject, the device comprising:
a body having a head for placing in contact with the skin of the subject;
vibration means for imparting a vibration to the head; and
means for modifying the frequency and/or amplitude of the vibrations of the head be passed to the skin of the subject.

41. The device according to claim 40, wherein the device is hand held.

42. The device according to claim 41, wherein the device may be held in and operated by a single hand of the user.

43. The device according to any of claims 40 to 42, wherein the head has an arcuate surface with arcs extending through an angle greater than 180°.

44. The device according to claim 43, wherein the head has an arcuate surface with arcs extending through an angle greater than 230°.

45. The device according to claim 44, wherein the head has an arcuate surface with arcs extending through an angle greater than 270°.

46. The device according to any of claims 43 to 45, wherein the surface of the head is a portion of a sphere greater, than a hemisphere.

47. The device according to claim 46, wherein the head is substantially spherical.

48. The device according to any of claims 40 to 47, wherein the body comprises an elongate portion, the head being located at the distal end of the elongate portion.

49. The device according to any of claims 40 to 48, wherein the head is integral with the body.

50. The device according to any of claims 40 to 49, wherein the means for modifying the vibrations of the head is a cover or coating applied to the head.

51. The device according to claim 50, wherein the cover or coating is removable.

52. The device according to either of claim 50 or 51, wherein the cover or coating is formed from a material that attenuates the vibrations transmitted by the head to the skin of the subject.

53. The device according to any of claims 40 to 52, wherein the vibration means, when activated, causes the entire device to vibrate.

54. The device according to any of claims 40 to 53, wherein the vibration means comprises an electric motor which, when activated, rotates an eccentrically mounted weight.

55. The device according to any of claims 40 to 54, further comprising a switch assembly, wherein the switch assembly is arranged to be activated by a lateral force applied by the user.

56. The device according to claim 55, wherein the switch assembly comprises a switch member to be pressed by the user.

57. The device according to claim 56, wherein the switch member is biased to the off position.

58. The device according to claim 57, wherein the switch member is a flexible, resilient plate.

59. A method for assessing the sensitivity of the skin of a subject comprising:
applying to the skin a device comprising a body and a head, the device being applied such that the head contacts the skin of the subject;
activating the device by applying a force to the device that extends laterally of the skin of the subject, such that activation does not require a force to be applied to the head of the device in the direction towards the skin of the subject, the head applying a vibrational stimulus to the skin of the subject when the device is activated; and
monitoring the response of the subject to the vibrational stimulus applied.

61. The use of a device according to any of claims 1 to 58 or claim 60 in providing vibrational stimuli to the body of a subject.

62. The use according to claim 61, for assessing the sensitivity of the skin of a subject.
